(19) United States
(12) Patent Application Publication
(10) Pub. No.: US 2010/0056482 A1
(43) Pub. Date: Mar. 4, 2010

(54) Calixarene Derivatives as Anticancer Agent

(75) Inventors: William Anthony Coleman, Caluire (FR); Loris Gilbert Baggetto, Villeurbanne (FR); Adina Nicoleta Lazar, Valcea (RO); Mickael Henri Michaud, Saint Just Saint Rambert (FR); Sandrine Magnard, La Valla En Gier (FR)

Correspondence Address: ARENT FOX LLP, 1050 CONNECTICUT AVENUE, N.W., SUITE 400, WASHINGTON, DC, 20036, US

(73) Assignees: CENTRE NATIONAL DE LA RECHERCHE SCIENTIFIQUE-CNRS, Paris Cedex 16 (FR); UNIVERITE CLAUDE BERNARD - LYON 1, Villeurbanne (FR)

(21) Appl. No.: 12/226,449

(22) PCT Filed: Apr. 18, 2007
(86) PCT No.: PCT/FR2007/051127

§ 371 Date: Oct. 8, 2009

(30) Foreign Application Priority Data
Apr. 18, 2006 (FR) 0603406

(51) Int. Cl. A61K 31/6615; A61P 35/00; A61P 35/02; C07F 9/09

(52) U.S. Cl. 514/107; 558/162

(57) ABSTRACT

This invention relates to compounds having the following formula (I):

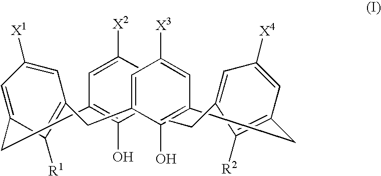

of compositions including them, uses of these compounds, particularly for preparation of medicines, and in vitro induction processes for apoptosis and direct death of cancer cells.

The present invention relates to the field of anticancer therapy. It particularly relates to specific calixarene derivatives exhibiting anticancer activity.

Cancers are one of the main causes of mortality and are in fact responsible for approximately 30% of deaths in France.

At the present time, one third of new cases of cancer exhibit resistance to multiple drugs (also referred to as “Multiple Drug Resistant” or MDR) or are chemoresistant. This resistance is a dramatic problem from the therapeutic point of view but also from a psychological point of view for patients.

Moreover, some compounds used for treating cancers, for example vinblastine, may cause a development of chemoresistant tumour cells and/or cause secondary effects, such as a certain degree of toxicity.

Finally, cancer treatments represent an economic problem, and in fact the cost of such treatment can be estimated at 15 billion euros in 2002.

There therefore remains a need for compounds having improved properties for treating cancer.

Surprisingly, the inventors have discovered that specific calixarene derivatives exhibit anticancer activity and make it possible to resolve, in whole or in part, the problems mentioned above.

According to a first aspect, an object of the invention is a compound having the following formula (I)

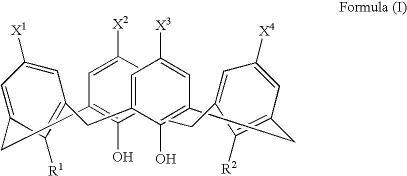

in which:
X¹, X², X³ and X⁴ represent, independently of one another, a hydrogen or halogen atom or an alkyl or a acyl group, in particular comprising from 1 to 10 carbon atoms, linear, branched or cyclic or aromatic;
R¹ and R² represent, independently of each other, —(R)ₓY, where
R represents an alkyl group comprising from 1 to 10 carbon atoms, alkene or alkyne comprising from 2 to 10 carbon atoms, linear, branched or cyclic, optionally substituted with one or more heteroatoms, in particular O, S, N or P, or R represents an aromatic radical, in particular comprising from 6 to 20 carbon atoms,
x represents 0 or 1, and
Y is a group chosen from the group comprising the phosphate, sulphate and carboxylic groups,
or one of its salts.

The compounds having formula (I) can be obtained according to techniques well known to persons skilled in the art. The synthesis of para-tertbutyl calyx[4]arene and the detertbutylation and functionalisation of top and bottom rings described in the literature by Gutsche, Shinkai and Kalchenko can for example be cited.

“Carboxylate group” means, within the meaning of the present invention, the chain —COOM, in which M can represent a hydrogen atom or a cation, in particular an alkali or alkaline-earth metal or an organic cation, such as ammonium, alkylammonium, phosphonium, guanidinium and pyridinium.

“Phosphate group” means the chain O—PO(OM)₂, in which M can be as defined above.

“Sulphate group” within the meaning of the present invention means the chain —SO₃M, in which M can be as defined above.

The salts of the compound having formula (I) can be obtained by techniques well known to persons skilled in the art.

In particular the compound according to the invention has formula (I) in which X¹, X², X³ and X⁴ represent the same atom or the same group.

The compound according to the invention can have formula (I) in which R¹ and R² represent the same group.

In particular, the compound according to the invention can have formula (I) in which R¹ and R² represent the same group, and X¹, X², X³ and X⁴ represent the same atom or the same group.

According to a particular embodiment the compound has formula (I) in which X¹, X², X³ and X⁴ represent, independently of one another, a halogen atom chosen from the group comprising the iodine, bromine, chorine and fluorine atom.

According to a particular embodiment the compound has formula (I) in which X¹, X², X³ and X⁴ represent a hydrogen atom.

According to a particular embodiment the compound has formula (I) in which X¹, X², X³ and X⁴ represent, independently of one another, an alkyl group chosen from the group comprising methyl, iso-propyl and tert-Butyl.

According to a particular embodiment the compound has formula (I) in which X¹, X², X³ and X⁴ represent, independently of one another, an acyl group chosen from the group comprising the methyl, iso-propyl and tert-Butyl groups.

In particular, the compound according to the invention has the following formula I(-a):

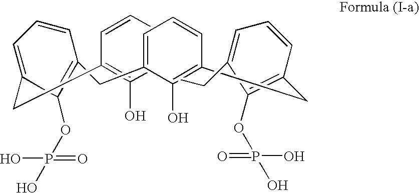

In particular, the compound according to the invention has the following formula (I-b):

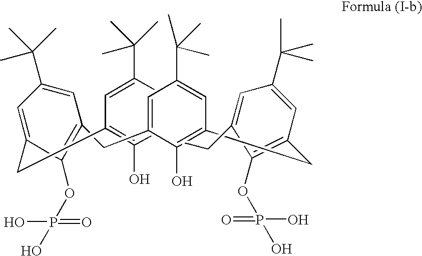

In particular, the compound according to the invention has the following formula (I-c):

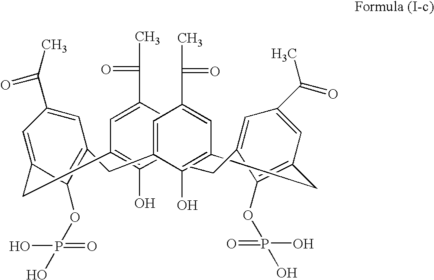

In particular, the compound according to the invention has the following formula (I-d):

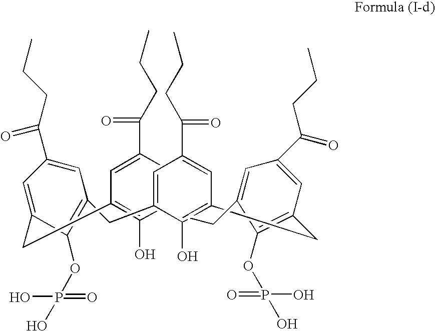

In particular, the compound according to the invention has the following formula (I-e):

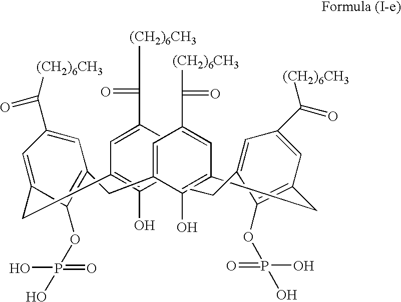

According to another aspect, another object of the invention is a complex compound comprising the association of several, in particular two, identical or different compounds as defined previously.

In particular, the compounds associated to form the complex compound are linked by covalent and/or non-covalent bonds.

The non-covalent bonds can be hydrogen bonds, ionic bonds, hydrophobic interactions, Van der Waals associations, aromatic-aromatic interactions and other electrostatic interactions.

When the compounds associated to form the complex compound are linked by covalent bonds, these compounds can be linked directly or via a spacer.

In particular, the spacer is a radical that is at least divalent, comprising 1 to 20 carbon atoms: diamides, diesters, ethylene glycol bridge.

According to another aspect, another object of the invention is a compound according to the invention or one of its pharmaceutically acceptable salts, as a pharmaceutical composition.

Within the meaning of the present invention, “Pharmaceutically acceptable salts” refers to salts appropriate for pharmaceutical use. By way of examples of pharmaceutically acceptable salts, the following can be cited: benzene sulphonate, bromohydrate, chlorhydrate, citrate, ethanesulphonate, fumarate, gluconate, iodate, isethionate, maleate, methanesulphonate, methylene-bis-b-oxynaphthoate, nitrate, nitrite, oxalate, palmoate, phosphate, salicylate, succinate, sulphate, tartrate, theophyllinacetate and p-toluenesulphate, in particular nitrate, nitrite, phosphate and especially ammonium.

The pharmaceutically acceptable salts of the compound according to the invention can be obtained by techniques well known to persons skilled in the art.

According to another of its aspects, another object of the invention is a pharmaceutical composition comprising at least one compound or at least one of its pharmaceutically acceptable salts, as defined previously.

The pharmaceutical composition according to the invention can further comprise at least one anti-tumour agent chosen from the group comprising:
angiogenesis inhibitors such as angiostatin, endostatin, genistein, staurosporin and thalidomide;
antiproliferative agents such as N-acetyl-D-sphingosin, aloe-emodin, apigenin, berberin hydrochloride, emodin, hydroxycholesterol and rapamycin;
DNA-synthesis inhibiting agents such as amethopterin, cytosine β-D-arabinofuranoside, 5-fluoro-5-deoxyuridine, ganciclovir, hydroxyurea, mercaptopurine and thioguanine;
enzyme inhibitors such as DL-aminoglutethimide, apicidin, 2′,4′,3,4-tetrahydroxychalcone, camptothecin, deguelin, depudecin, doxycyclin, etoposide, formestane, fostriecine, hispidine, indomethacin, mevinolin, oxamflatin, roscovitine, trichostatin and tyrphostin AG.

“Anti-tumour agent” means, within the meaning of the present invention, a compound for combating and/or preventing cancer.

The pharmaceutical composition can comprise the compound according to the invention and the anti-tumour agent in a molar ratio ranging from 10/1 to 1/10.

The pharmaceutical compositions according to the invention can be administered by different routes.

By way of examples of administration routes, the following routes can be cited: oral, rectal, cutaneous, pulmonary, nasal, sublingual, the parenteral route, in particular intradermic, subcutaneous, intramuscular, intravenous, intra-arterial, intra-rachidian, intra-articular, intrapleural, intraperitoneal, ocular, inhalations, transdermic, epidural, intrabronchial, intrabursal, intracameral, intracardiac, intracerebral, intracavernous, intracerebroventricular, intracisternal, intragastric, intralesional, intralymphatic, intraosseous, intraspinal, intrathecal, intratracheal, intraduodenal, intra tympanic, intrarethal, intra-uterine, intravaginal, intravesical, intravitreal, sublabial, rectal, subconjunctival, retrobulbar, intratumoral in particular subconjunctival, retrobulbar and intratumoral and especially intratumoral.

The pharmaceutical compositions according to the invention can be administered one or more times or in sustained release.

The pharmaceutical compositions according to the invention can be in different forms, in particular in a form chosen from the group comprising tablets, capsules, pills, syrups, suspensions, solutions, powders, granules, emulsions, microspheres and injectable solutions and solid lipid nanoparticles, in particular solid lipid nanoparticles.

These different forms can be obtained by techniques well known to persons skilled in the art.

In particular, the formulations appropriate for administration by parenteral route, the pharmaceutically acceptable carriers appropriate for this administration route and the corresponding formulation and administration techniques can be implemented according to methods well known to persons skilled in the art, in particular those described in the manual Remington's Pharmaceutical Sciences (Mack Publishing, Easton, Pa, 20^{th} edition, 2000).

In a particular embodiment, the compound according to the invention or at least one of its pharmaceutically acceptable salts is present in the pharmaceutical composition in a quantity ranging from 1 to 400 mg per unit dose, and in particular from 10 to 40 mg.

The pharmaceutical composition according to the invention can be administered in one or more doses per day, in particular 1 to 3 doses per day.

Advantageously, the compound can be administered in a quantity ranging from 0.1 to 6 mg per day and per kg.

Advantageously, the pharmaceutical composition comprises a quantity of at least one compound or at least one of its pharmaceutically acceptable salts ranging from 1 to 100 mg, in particular from 10 to 40 mg.

The pharmaceutical composition can also comprise a pharmaceutically acceptable carrier.

“Pharmaceutically acceptable carrier” means, within the meaning of the present invention, a material that is appropriate to use in a pharmaceutical product.

By way of examples of pharmaceutically acceptable carrier, lactose, starch, optionally modified, cellulose, hydroxypropyl cellulose, hydroxypropylmethyl cellulose, mannitol, sorbitol, xylitol, dextrose, calcium sulphate, calcium phosphate, calcium lactate, dextrates, inositol, calcium carbonate, glycin, bentonite, polyvinylpyrrolidone, and mixtures thereof, can be cited.

The pharmaceutical composition can comprise a proportion of pharmaceutically acceptable carrier ranging from 5 to 99% by weight, more particularly from 10 to 90% by weight, and especially from 20 to 75% by weight with respect to the total weight of the composition.

According to another aspect, another object of the invention is the use of a compound or one of its pharmaceutically acceptable salts as defined above for manufacturing a pharmaceutical composition intended for the treatment of cancers, whatever their nature and their degree of anaplasia, in particular melanomas, carcinomas, sarcomas, fibrosarcomas, leukaemias, lymphomas, neuroblastomas, medulloblastomas, glioblsatomas, astrocytomas, angioblastomas, meningiomas, retinoblastomas, prolactinomas, macrobulimia, leiomyosarcomas, mesotheliomas, choriocarcinomas, pheochromocytomas, myelomas, polycythemias, angiosarcomas, extra-skeletal chondrosarcomas, hemangiosarcomas, osteosarcomas, chondrosarcomas and especially melanomas, carcinomas, sarcomas, fibrosarcomas and leukaemias.

By way of examples of such cancers, the following can be cited: pancreatic cancer, cancers of the oropharynx, stomach cancer, cancer of the oesophagus, colon and rectal cancer, brain cancer, in particular gliomas, ovarian cancer, liver cancer, kidney cancer, cancer of the larynx, thyroid cancer, lung cancer, bone cancer, multiple myelomas, mesotheliomas and melanomas, skin cancer, breast cancer, prostate cancer, bladder cancer, cancer of the uterus, testicular cancer, non-Hodgkin's lymphoma, leukaemia, Hodgkin's disease, cancer of the tongue, cancer of the duodenum, bronchial cancer, pancreatic cancer and soft tissue cancers, as well as metastatic secondary locations of the aforementioned cancers, such as in the lung, liver and breast.

According to another particular embodiment of the invention, the compound is associated with at least one anti-tumour agent chosen from the group comprising:
angiogenesis inhibitors such as angiostatin, endostatin, genistein, staurosporin and thalidomide;
antiproliferative agents such as N-acetyl-D-sphingosine, aloe-emodin, apigenin, berberin hydrochloride, hydroxycholesterol and rapamycin;
DNA-synthesis inhibiting agents such as amethopterin, cytosine β-D-arabinofuranoside, 5-fluoro-5-deoxyuridine, ganciclovir, hydroxyurea, mercaptopurine and thioguanine;
enzyme inhibitors such as DL-aminoglutethimide, apicidin, 2′,4′,3,4-tetrahydroxychalcone, camptothecin, deguelin, depudecin, doxycylin, etoposide, formestane, fostriecine, hispidine, indomethacin, mevinolin, oxamflatin, roscovitine, trichostatin and tyrphostin AG.

“Associated” means the fact that the compound and the anti-tumour agent can be administered simultaneously, separately or staged over time.

Another object of the present invention is a method for the in vitro induction of apoptosis of cancerous cells, comprising putting said cells in the presence of at least one compound according to the invention.

Other advantages and characteristics of the invention will become apparent in light of the following figures and examples.

The following figures and examples are given by way of illustration and non-limitatively.

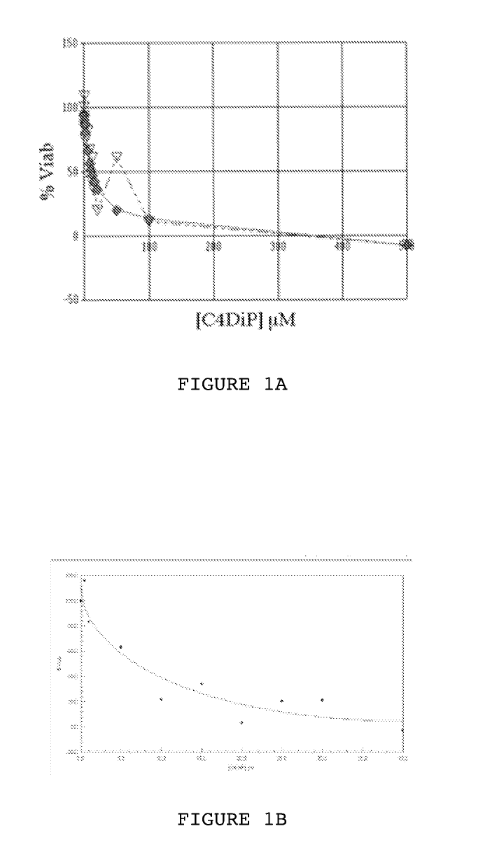

FIG. 1 (A and B) illustrates, in the form of curves, the effect of calyx[4]arene dihydrophosphonic acid on a culture of chemosensitive human acute lymphoblastic leukaemic cells (CEM/s) by measuring the cell viability. In FIG. 1B, the root of the regression equation is: a+b.x^{1.5}+c.x^{0.5} in which X to Y=5.000000000000E+01 and X=6.874748783580E+00.

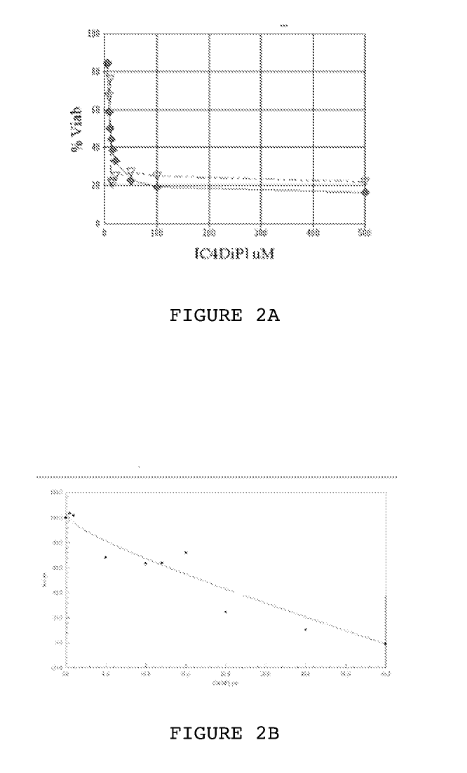

FIG. 2 (A and B) illustrates, in the form of curves, the effect of calyx[4]arene dihydrophosphonic acid on a culture of chemosensitive human acute lymphoblastic leukaemic cells (CEM/s) by measuring the cell viability. In FIG. 2B, the root of the regression equation is: a+b.x+c.x^{0.5} in which X to Y=5.000000000000E+01 and X=1.701434371714E+01.

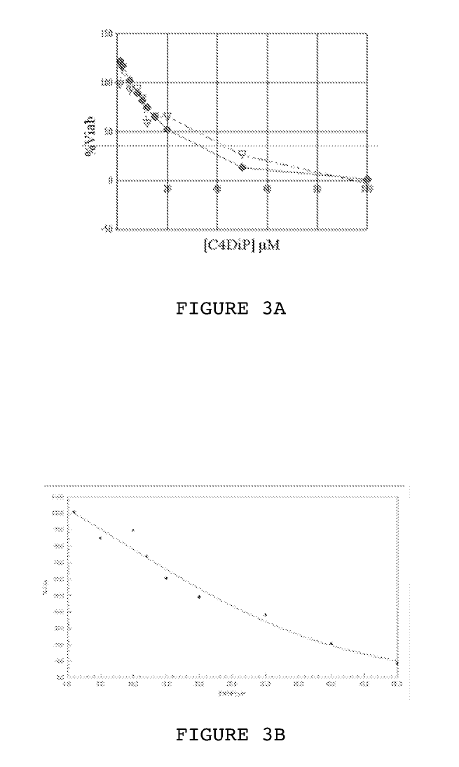

FIG. 3 (A and B) illustrates, in the form of curves, the effect of calyx[4]arene dihydrophosphonic acid on a culture of human choroid melanoma cells (MU2), by measuring the cell viability. In FIG. 3B, the root of the regression equation is: a.exp(b−(x−b)²)/(2.c²)) in which X to Y=5.000000000000E+01 and X=2.189932229650E+01.

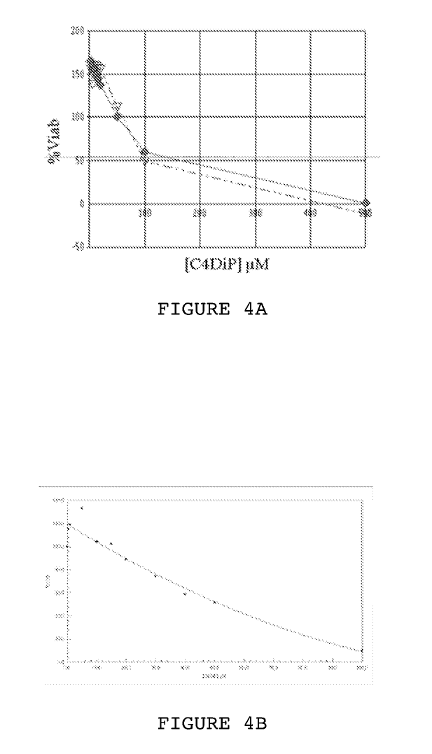

FIG. 4 (A and B) illustrates, in the form of curves, the effect of calyx[4]arene dihydrophosphonic acid on a culture of human choroid melanoma cells (MU2F), by measuring the cell viability. In FIG. 4B, the root of the regression equation is: ax²+b.x+c in which X to Y=5.000000000000E+01 and X=5.209011013695E+01.

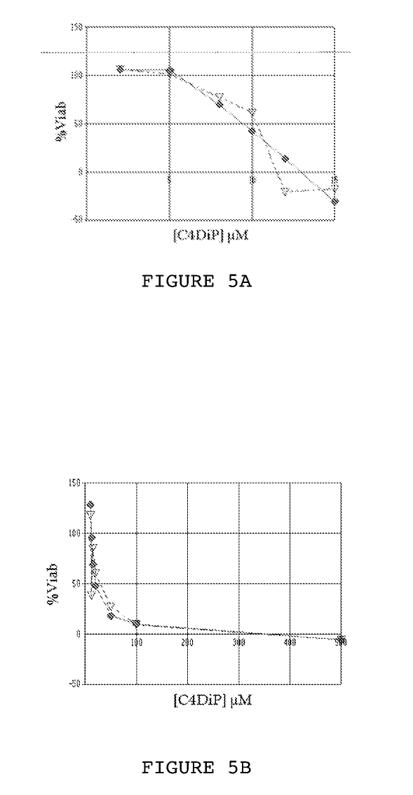

FIG. 5 (A and B) illustrates, in the form of curves, the effect of calyx[4]arene dihydrophosphonic acid on a culture of human fibrosarcoma cells (HT1080), by measuring the cell viability.

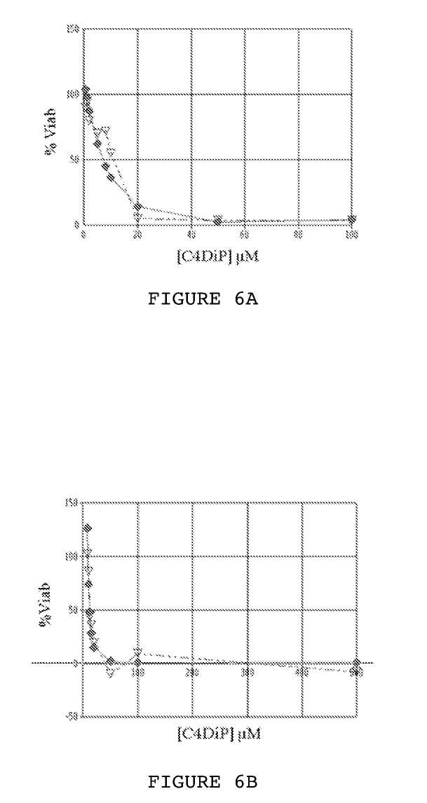

FIG. 6 (A and B) illustrates, in the form of curves, the effect of calyx[4]arene dihydrophosphonic acid on a culture of human choroid melanoma cells (SP6.5), by measuring the cell viability.

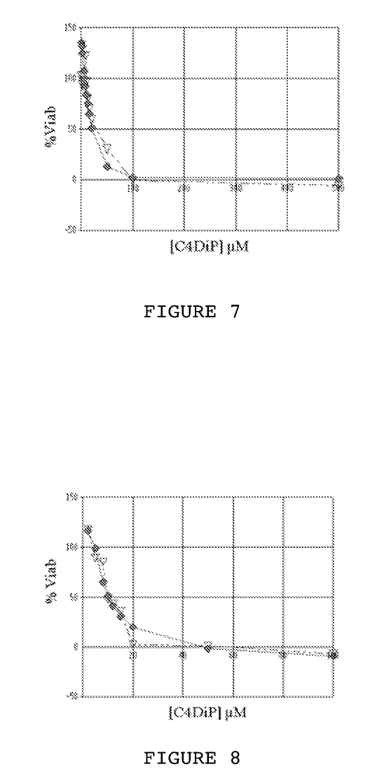

FIG. 7 illustrates, in the form of curves, the effect of calyx[4]arene dihydrophosphonic acid on a culture of human iris melanoma cells (IPC227), by measuring the cell viability.

FIG. 8 illustrates, in the form of curves, the effect of calyx[4]arene dihydrophosphonic acid on a culture of human skin melanoma cells (MEWO), by measuring the cell viability.

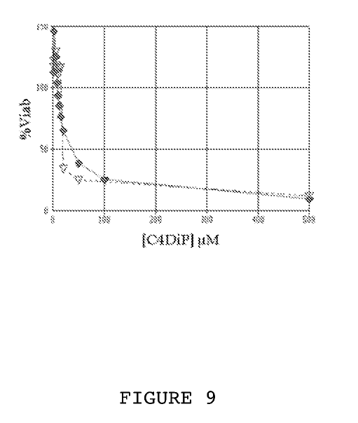

FIG. 9 illustrates in the form of curves, the effect of calyx[4]arene dihydrophosphonic acid on a culture of human uveal melanoma cells (DLM.1), by measuring the cell viability.

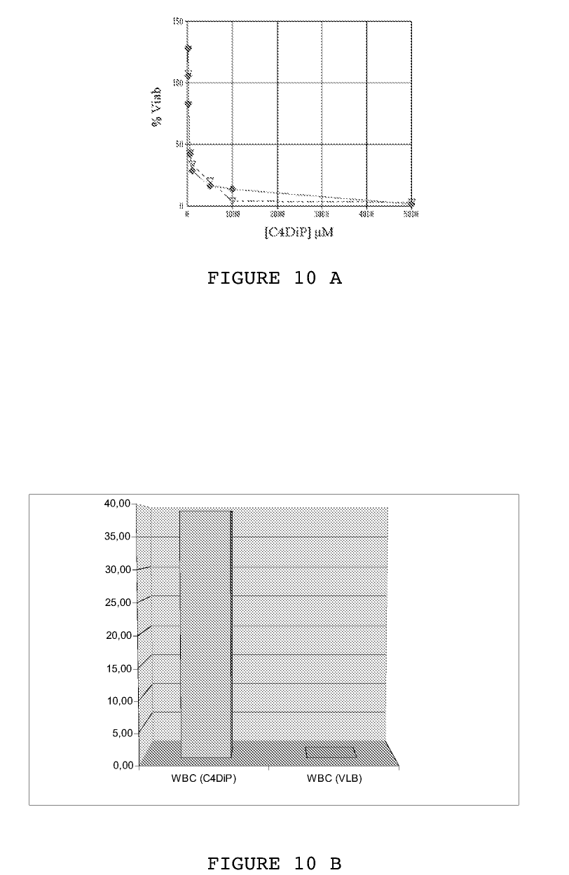

FIG. 10 (A and B) illustrates, in the form of curves, the effect of calyx[4]arene dihydrophosphonic acid and that of vinblastin (VLB) on a primary culture of human PBMC (peripheral blood mononuclear cells) by measuring the cell viability.

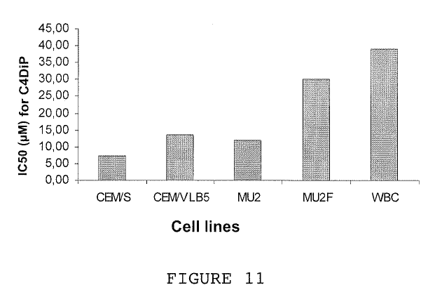

FIG. 11 illustrates, in the form of a histogram, the anticancer and lymphoprotective effect of calyx[4]arene dihydrophosphonic acid (C4Dip), by representing the mean IC₅₀ for different cell cultures.

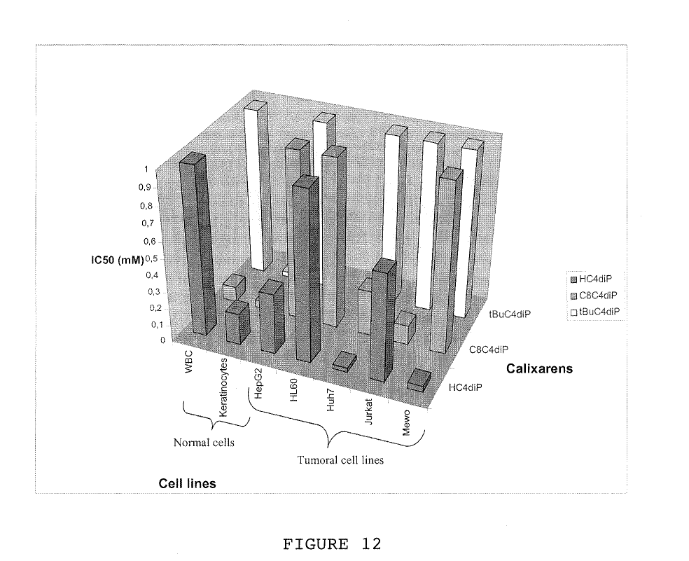

FIG. 12 illustrates, in the form of histogram, the anticancer effect of calyx[4]arene dihydroxyphosphonic acid (C4diP), tert-butyl-calyx[4]arene dihydroxyphosphonic acid (tBuC4diP) (formula I-b) and para-octanoyl-calyx[4]arene dihydroxyphosphonic acid (C8C4diP) (formula I-e), by representation of the mean IC₅₀ for different cell cultures.

### EXAMPLES

### I. Example 1

### Study of the Anticancer Effect of Calyx[4]arene Dihydrophosphonic Acid (C4diP) on Cell Cultures

This example illustrates the anticancer effect of calyx[4]arene dihydrophosphonic acid (C4diP) on different tumour cells in culture, in particular leukaemic, melanoma and fibrosarcoma cells.

### I.1 Experimental Protocol

The cells (0.2×10⁶/ml) were incubated in a sterile fashion in 100 μl of culture medium (RPMI-1640 supplemented with 10% foetal calf serum, penicillin, streptomycin and amphotericin B) at day DO with variable quantities (from 0 to 100 mM) of C4diP at 37° C. and 5% CO₂ in a water-saturated atmosphere. At the 4^{th} day, 20 μl of MTS (non-radioactive cell proliferation test (Promega Cat. #: G 3580) is added to the previous 100 μl and then a spectrophotometric reading is carried out at 490 nm after 2 hours of incubation against reagent blanks.

The cell lines used are as follows:
CEM: human acute lymphoblastic leukaemia. Cells of the T lymphoblast type.
CEM/s: chemosensitive CEM.
CEM/VLB5: chemoresistant CEM.

Chemoresistance was initially induced by the presence of vinblastin in the cell culture medium at increasing concentrations. In the case of CEM/VLB5, chemoresistance is maintained in culture in the presence of vinblastin at a concentration of 5 μg/ml.
MU2: human choroid melanoma created at the Institut de Biologie et Chimie des Proteines (IBCP, Lyon, France).
MU2F: human choroid melanoma which, implanted in mice, causes hepatic metastasis.
HT1080: human fibrosarcoma.
SP6.5: human choroid melanoma.
IPC227: human iris melanoma.
MEWO: human skin melanoma.
DLM.1: human uveal melanoma.

The anticancer effect of C4diP is evaluated by measuring the cell viability as a function of variable quantities of C4diP.

In order to determine the anticancer effect of C4diP on a particular culture of tumour cells, the IC₅₀ was calculated. The IC₅₀ corresponds to the mean concentration of C4diP at which the cells exhibit a 50% mortality.

### I.2 Results

I.2.1 Anticancer Effect of C4diP on a Culture of Chemosensitive cells: CEM/s

The effect of C4diP on a culture of chemosensitive human acute lymphoblastic leukaemic cells is presented in FIG. 1 (A and B).

The IC₅₀ measurement of C4diP shows a mortality of 50% for a mean concentration of 7.33 μM of C4diP with a standard deviation of 3.06.

This experiment shows clearly that C4diP makes it possible to combat chemosensitive cancer cells. C4diP is therefore an effective compound for treating cancers.

### I.2.2 Anticancer Effect of C4diP on a Culture of Chemoresistant Tumour Cells: CEM/VLB5

The effect of C4diP on a culture of chemoresistant human acute lymphoblastic leukaemic cells is presented in FIG. 2 (A and B).

The IC₅₀ measurement of C4diP shows a 50% mortality for a mean concentration of 13.55 μM of C4diP with a standard deviation of 4.88.

This experiment shows clearly that C4diP makes it possible to combat chemoresistant cancer cells. C4diP is therefore an effective compound for treating cancers of the Multiple Drug Resistant (MDR) type, for which the compounds conventionally used are not effective, such as vinblastin.

### I.2.3 Anticancer Effect of C4diP on Various Cultures of Tumour Cells: MU2, MU2F, HT1080, SP6.5, IPC227, MEWO, DLM.1

The effect of C4diP on various cultures of melanoma and fibrosarcoma tumour cells is presented in FIGS. 3 to 9.

The IC₅₀ measurement of C4diP relating to these various cell lines is presented in the following table:

| Cell Line | IC₅₀ in μM | Standard deviation |
|---|---|---|
| MU 2 | 12.06 | 5.96 |
| MU2F | 32.10 | 17.27 |
| HT1080 | 14.36 | Not determinable |
| SP6.5 | 9.4 | Not determinable |
| IPC227 | 20.2 | Not determinable |
| MEWO | 10.2 | Not determinable |
| DLM.1 | 30.83 | Not determinable |

This experiment shows clearly that C4diP makes it possible to combat various types of cancer cells. C4diP is therefore an effective compound for treating various types of cancer.

### II. Example 2

### Comparative Study of the Anticancer Effect of calyx[4]arene dihydrophosphonic acid (C4diP), tert-butyl-calyx[4]arene dihydroxyphosphonic acid (tBuC4diP) and para-octanoyl-calyx[4]arene dihydroxyphosphonic acid (C8C4diP) on Cell Cultures

This example illustrates the anticancer effect of calyx[4]arene dihydrophosphonic acid (C4diP), tert-butyl-calyx[4]arene dihydroxyphosphonic acid (tBuC4diP) and para-octanoyl-calyx[4]arene dihydroxyphosphonic acid (C8C4diP) on various tumour cells in culture, in particular leukaemic, melanoma, human hepatoma and hepatocellular carcinoma cells.

### II.1 Experimental Protocol

The cells (0.2×10⁶/ml) were incubated in a sterile fashion in 100 μl of culture medium (RPMI-1640 supplemented with 10% foetal calf serum, penicillin, streptomycin and amphotericin B) at day DO with variable quantities (from 0 to 100 mM) of C4diP, tBuC4diP or C8C4diP at 37° C. and 5% CO₂ in a water-saturated atmosphere. On the 3rd day, 20 μl of MTS (non radioactive cell proliferation test (Promega Cat. #: G 3580) are added to the previous 100 μl and then a spectrophotometric reading is carried out at 490 nm after 2 hours of incubation against reagent blanks.

The cell lines used are as follows:
keratinocytes
WBC: leucocytes
MEWO: human skin melanoma
jurkat: T-cell acute lymphoblastic leukaemia. Cells of the T-lymphocyte type.
HL-60: promyelocytary acute leukaemia. Cells of the promyeloblast type.
Huh7: human hepatoma
Hep-G2: hepatocellular carcinoma.

The anticancerous effect of C4diP, tBuC4diP and C8C4diP is evaluated by measuring cell viability as a function of variable quantities of these compounds.

In order to determine the anticancer effect of C4diP, tBuC4diP and C8C4diP on a particular tumour cell culture, the IC₅₀ was calculated. The IC₅₀ corresponds to the mean concentration of C4diP, tBuC4diP or C8C4diP at which the cells exhibit a 50% mortality.

### II.2 Results

II.2.1 Anticancer Effect of C4diP, tBuC4diP and C8CdiP on Various Tumour Cell Cultures: jurkat, MEWO, Hl-60, Huh7 and Hep-G2

The effects of the three calix-arenes C4diP, tBuC4diP and C8C4diP on the various tumour cell cultures are presented in FIG. 12.

The IC₅₀ measurement for C4diP, tBuC4diP and C8C4diP relating to these various cell lines is presented in the following table:

| | | IC₅₀ in mM | | |
|---|---|---|---|---|
| Cell line | | tBuC4diP | C8C4diP | C4diP |
| Non | WBC | 1 | 0.095 | 1 |
| tumoral | (leucocytes) | | | |
| cells | Keratonicytes | 0.031 | 0.046 | 0.185 |
| Tumoral | HepG2 | 1 | 1 | 0.36 |
| cells | HL-60 | Not determined | 1 | 1.33 |
| | Huh 7 | 1 | 0.27 | 0.027 |
| | Jurkat | 1 | 0.113 | 0.635 |
| | Mewo | 1 | 1 | 0.038 |

This experiment shows clearly that C4diP, tBuC5diP and C8C4diP make it possible to combat various types of cancer cells. C4diP, tBuC5diP and C8C4diP are therefore effective compounds for treating various types of cancer.

### III. Example 3

### Comparison of the Effect of calyx[4]arene dihydrophosphonic acid (C4diP and Vinblastin on a PBMC Cell Culture

This example illustrates the effect of calyx[4]arene dihydrophosphonic (C4diP) on a human PBMC primary culture. This effect is compared with that of vinblastin.

### III.1 Experimental Protocol

The protocol used is that described previously in example 1. When vinblastin is used in the culture medium, it replaces C4diP.

### III.2 Lymphoprotective Effect of C4diP

The effect of C4diP and vinblastin on a primary culture of human PBMC (peripheral blood mononuclear cells is presented in FIG. 10 (A and B).

The IC₅₀ measurement of C4diP shows a 50% mortality for a mean concentration of 39.1 μM of C4diP.

A comparison between the IC₅₀ of C4diP and of vinblastin on primary cultures of PBMC (WBC=White Blood Cell) shows a good lymphoprotective effect of C4diP (IC₅₀=39 μM) compared with vinblastin (IC₅₀=0.005 μM),

### IV Example 4

### Anticancer and Lymphoprotective Effect of calyx[4]arene dihydrophosphonic (C4diP)

A graphical representation of the mean IC₅₀ of C4diP is presented in FIG. 11. The area of activity of C4diP is situated between 7 and 15 μM, without any discrimination between MDR chemoresistant and chemosensitive cells, with the exception of the MU2F metastasis, which seems more resistant to C4diP than the other cell lines tested.

However, it is interesting to note that the IC₅₀ of C4diP is 39 μM for healthy lymphocytes, which shows a good lymphoprotective effect of C4diP vis-à-vis the other more sensitive lines tested.

These results show clearly that some calixarene derivatives such as C4diP exhibit an antitumour activity on tumoural cell lines maintained in culture, in particular in the case of the human acute lymphoblastic leukaemia CEM and its chemoresistant derivative CEM/VLB5, as well as on the MU2 human uveal melanoma cell line.

1-20. (canceled)

21. A pharmaceutical composition comprising at least one compound having the following formula (I): 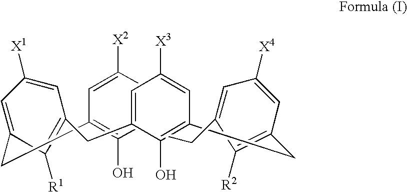
or at least one of its pharmaceutically acceptable salts;
wherein:
X¹, X², X³ and X⁴ represent, independently of one another, a hydrogen or halogen atom or a linear, branched or cyclic C₁₋₁₀ alkyl or acyl group;
R¹ and R² represent, independently of each other, -(R)ₓY, wherein
R represents a linear, branched or cyclic C₁₋₁₀ alkyl, C₂₋₁₀ alkenyl or C₂₋₁₀ alkynyl group, optionally substituted with one or more heteroatoms, or R represents a C₆₋₂₀ aromatic radical,
x represent 0 or 1, and
Y is a phosphate, sulphate or carboxylic group.

22. The composition of claim 19, wherein X¹, X², X³ and X⁴ represent the same atom or the same group.

23. The composition of claim 20, wherein X¹, X², X³ and X⁴ each represents a hydrogen atom.

24. The composition of claim 19, wherein R¹ and R² represent the same group.

25. The composition of claim 19, wherein X¹, X², X³ and X⁴ represent, independently of one another, an iodine, bromine, chlorine or fluorine atom.

26. The composition of claim 19, wherein X¹, X², X³ and X⁴ represent, independently of one another, methyl, iso-propyl or tert-butyl.

27. The composition of claim 19, wherein said at least one compound has the following formula (I-a)

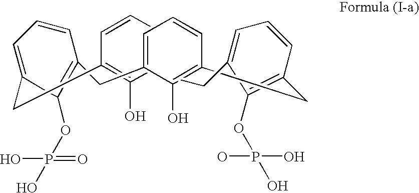

28. The composition of claim 19, wherein said at least one compound has the following formula (I-b):

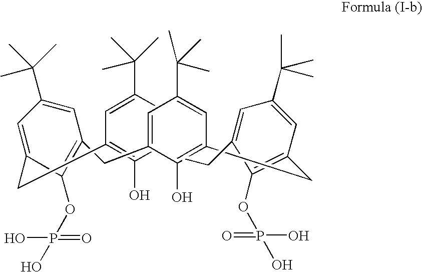

29. The composition of claim 19, wherein said at least one compound has the following formula (I-c):

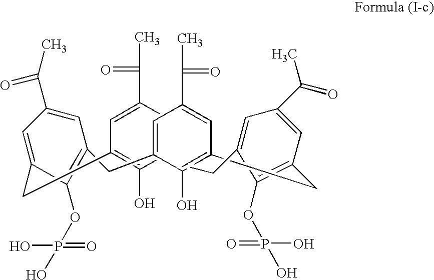

30. The composition of claim 19, wherein said at least one compound has the following formula (I-d):

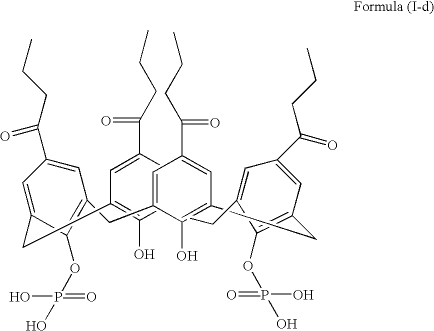

31. The composition of claim 19, further comprising at least one anti-tumour agent, wherein the anti-tumour agent is an angiogenesis inhibitor, an antiproliferative agent, a DNA-synthesis inhibiting agent or an enzyme inhibitor.

32. The composition of claim 29, wherein:
the angiogenesis inhibitor is angiostatin, endostatin, genistein, staurosporin or thalidomide;
the antiproliferative agent is N-acetyl-D-sphingosin, aloe-emodin, apigenin, berberin hydrochloride, emodin, hydroxycholesterol or rapamycin;
the DNA-synthesis inhibiting agent is amethopterin, cytosine β-D-arabinofuranoside, 5-fluoro-5-deoxyuridine, ganciclovir, hydroxyurea, mercaptopurine or thioguanine; and
the enzyme inhibitor is DL-aminoglutethimide, apicidin, 2′,4′,3,4-tetrahydroxychalcone, camptothecin, deguelin, depudecin, doxycyclin, etoposide, formestane, fostriecine, hispidine, indomethacin, mevinolin, oxamflatin, roscovitine, trichostatin or tryphostine AG.

33. A method of treating cancer comprising administering to a subject in need thereof a therapeutically effective amount of a composition of claim 1.

34. The method of claim 31, wherein the composition is administered by at least one route selected from oral, rectal, cutaneous, pulmonary, nasal, sublingual, the parenteral route, in particular intradermic, subcutaneous, intramuscular, intravenous, intra-arterial, intra-rachidian, intra-articular, intrapleural, intraperitoneal, ocular, inhalations, transdermic, epidural, intrabronchial, intrabursal, intracameral, intracardiac, intracerebral, intracavernous, intracerebroventricular, intracisternal, intragastric, intralesional, intralymphatic, intraosseous, intraspinal, intrathecal, intratracheal, intraduodenal, intra tympanic, intrarethal, intra-uterine, intravaginal, intravesical, intravitreal, sublabial, rectal, subconjunctival, retrobulbar or intratumoral administration.

35. The method of claim 32, wherein the composition is administered intratumorally.

36. The method of claim 31, wherein the composition is in a form of a tablet, capsule, pill, syrup, suspension, solution, powder, granule, emulsion, microsphere, injectable solution or solid lipid nanoparticles.

37. The method of claim 34, wherein the composition is in a form of solid lipid nanoparticles.

38. The method of claim 31, wherein the cancer is melanoma, carcinoma, sarcoma, fibrosarcoma, leukaemia, lymphoma, neuroblastoma, medulloblastoma, glioblastoma, astrocytoma, angioblastoma, meningioma, retinoblastoma, prolactinoma, macrobulimia, leiomyosarcoma, mesothelioma, choriocarcinoma, pheochromocytoma, myeloma, polycythemia, angiosarcoma, extra-skeletal chondrosarcoma, hemangiosarcoma, osteosarcoma or chondrosarcoma.

39. The method of claim 36, wherein the cancer is melanoma, carcinoma, sarcoma, fibrosarcoma or leukaemia.

40. The method of claim 31, wherein the compound is associated with at least one anti-tumour agent, wherein the anti-tumour agent is an angiogenesis inhibitor, an antiproliferative agent, a DNA-synthesis inhibiting agent or an enzyme inhibitor.

41. The method of claim 38, wherein:
the angiogenesis inhibitor is angiostatin, endostatin, genistein, staurosporin or thalidomide;
the antiproliferative agent is N-acetyl-D-sphingosin, aloe-emodin, apigenin, berberin hydrochloride, emodin, hydroxycholesterol or rapamycin;
the DNA-synthesis inhibiting agent is amethopterin, cytosine β-D-arabinofuranoside, 5-fluoro-5-deoxyuridine, ganciclovir, hydroxyurea, mercaptopurine or thioguanine; and
the enzyme inhibitor is DL-aminoglutethimide, apicidin, 2′,4′,3,4-tetrahydroxychalcone, camptothecin, deguelin, depudecin, doxycyclin, etoposide, formestane, fostriecine, hispidine, indomethacin, mevinolin, oxamflatin, roscovitine, trichostatin or tryphostine AG.

42. An in vitro method for inducing apoptosis of cancerous cells, comprising putting said cells in the presence of at least one compound as defined in claim 1.

43. A compound having the following formula (I-c):

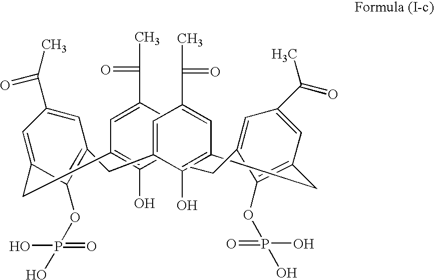

44. A compound having the following formula (I-d): [chemical structure]
